(19) United States
(12) Patent Application Publication
(10) Pub. No.: US 2024/0173219 A1
(43) Pub. Date: May 30, 2024

(54) Lotion Composition

(71) Applicants: Latif Jackson, Stillwater, OK (US); Lawrence Jackson, Stillwater, OK (US)

(72) Inventors: Latif Jackson, Stillwater, OK (US); Lawrence Jackson, Stillwater, OK (US)

(21) Appl. No.: 18/158,537

(22) Filed: Jan. 24, 2023

Related U.S. Application Data
(60) Provisional application No. 63/428,437, filed on Nov. 29, 2022.

(51) Int. Cl. A61K 8/19; A61K 8/06; A61K 8/34; A61K 8/36; A61K 8/44; A61K 8/89; A61K 8/9794

(52) U.S. Cl. CPC A61K 8/19; A61K 8/06; A61K 8/345; A61K 8/36; A61K 8/44; A61K 8/89; A61K 8/9794

(57) ABSTRACT

The present invention relates to a lotion compound that moisturizes and sanitizes the hands of a user simultaneously. The compound is preferably comprised of purified water, propylene glycerol, glycerin, stearic acid, cetyl alcohol, disodium EDTA, glyceryl stearate, aloe, barbadensis leaf juice, triethanolamine, white petroleum, dimethicone, propylparaben, methylparaben, allantoin, carbomer, propylene, diazolidinyl urea, and iodopropynyl butylcarbamate, each at 1% weight.

### CROSS-REFERENCE TO RELATED APPLICATION

The present application claims priority to, and the benefit of, U.S. Provisional Application No. 63/428,437, which was filed on Nov. 29, 2022, and is incorporated herein by reference in its entirety.

### FIELD OF THE INVENTION

The present invention relates generally to the field of skin lotions. More specifically, the present invention relates to a lotion compound that moisturizes and sanitizes the hands of a user simultaneously. The compound is preferably comprised of purified water, propylene glycerol, glycerin, stearic acid, cetyl alcohol, disodium EDTA, glyceryl stearate, aloe, barbadensis leafjuice, triethanolamine, white petroleum, dimethicone, propylparaben, methylparaben, allantoin, carbomer, propylene, diazolidinyl urea, and iodopropynyl butylcarbamate, each at 1% weight. Accordingly, the present disclosure makes specific reference thereto. Nonetheless, it is to be appreciated that aspects of the present invention are also equally applicable to other like applications, devices, and methods of manufacture.

### BACKGROUND

Existing skin lotion known in the art is one-dimensional and only moisturizes the skin of the user. Further, existing skin lotion known in the art does not sanitize the skin of germs. As a result, a skin sanitizer must be used. However, skin sanitizers are known to dry out the skin, and can be painful to apply to open wounds on the skin.

Therefore, there exists a long-felt need in the art for an improved skin lotion. There also exists a long-felt need in the art for a lotion compound that moisturizes skin. Further, there exists a long-felt need in the art for a lotion compound that also sanitizes skin of germs. More specifically, there exists a long-felt need in the art for a lotion compound simultaneously sanitizes and moisturizes skin.

The subject matter disclosed and claimed herein, in one embodiment thereof, comprises a lotion compound. The compound moisturizes and sanitizes the hands of a user simultaneously. The compound is preferably comprised of purified water, propylene glycerol, glycerin, stearic acid, cetyl alcohol, disodium EDTA, glyceryl stearate, aloe, barbadensis leaf juice, triethanolamine, white petroleum, dimethicone, propylparaben, methylparaben, allantoin, carbomer, propylene, diazolidinyl urea, and iodopropynyl butylcarbamate, each at 1% weight.

In this manner, the lotion compound of the present invention accomplishes all the foregoing objectives and provides an improved skin lotion. The lotion compound simultaneously sanitizes and moisturizes skin during use. In this manner, the lotion compound overcomes the limitations and disadvantages of existing skin lotion and sanitizing products known in the art.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a lotion compound for human skin. The compound can be used to simultaneously moisturize and sanitize the hands of a user. The compound is comprised of a plurality of ingredients. Said ingredients include purified water, propylene glycerol, glycerin, stearic acid, cetyl alcohol, disodium EDTA, glyceryl stearate, aloe, barbadensis leaf juice, triethanolamine, white petroleum, dimethicone, propylparaben, methylparaben, allantoin, carbomer, propylene, diazolidinyl urea, and iodopropynyl butylcarbamate. In the preferred embodiment, each ingredient makes up the lotion compound at 1% weight. However, the percentage weights of each ingredient may differ in various embodiments of the compound.

Accordingly, the lotion compound of the present invention is particularly advantageous as it provides an improved skin lotion that simultaneously sanitizes and moisturizes skin during use. As a result, the lotion compound overcomes the limitations of existing moisturizers known in the art. Further, the lotion compound overcomes the disadvantages of existing skin sanitizers known in the art.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a graphical view of ingredients of one potential embodiment of a lotion compound of the present invention in accordance with the disclosed architecture;

FIG. 2 illustrates a perspective view of a container of one potential embodiment of a lotion compound of the present invention in accordance with the disclosed architecture; and

FIG. 3 illustrates a perspective view of a container of one potential embodiment of a lotion compound of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved skin lotion. There also exists a long-felt need in the art for a lotion compound that moisturizes skin. Further, there exists a long-felt need in the art for a lotion compound that also sanitizes skin of germs. More specifically, there exists a long-felt need in the art for a lotion compound simultaneously sanitizes and moisturizes skin.

The present invention, in one exemplary embodiment, is comprised of a lotion compound that can simultaneously moisturize and sanitize the hands of a user. The compound is comprised of a plurality of ingredients. Said ingredients include purified water, propylene glycerol, glycerin, stearic acid, cetyl alcohol, disodium EDTA, glyceryl stearate, aloe, barbadensis leaf juice, triethanolamine, white petroleum, dimethicone, propylparaben, methylparaben, allantoin, carbomer, propylene, diazolidinyl urea, and iodopropynyl butylcarbamate. In the preferred embodiment, each ingredient makes up the lotion compound at 1% weight. However, the percentage weights of each ingredient may differ in various embodiments of the compound.

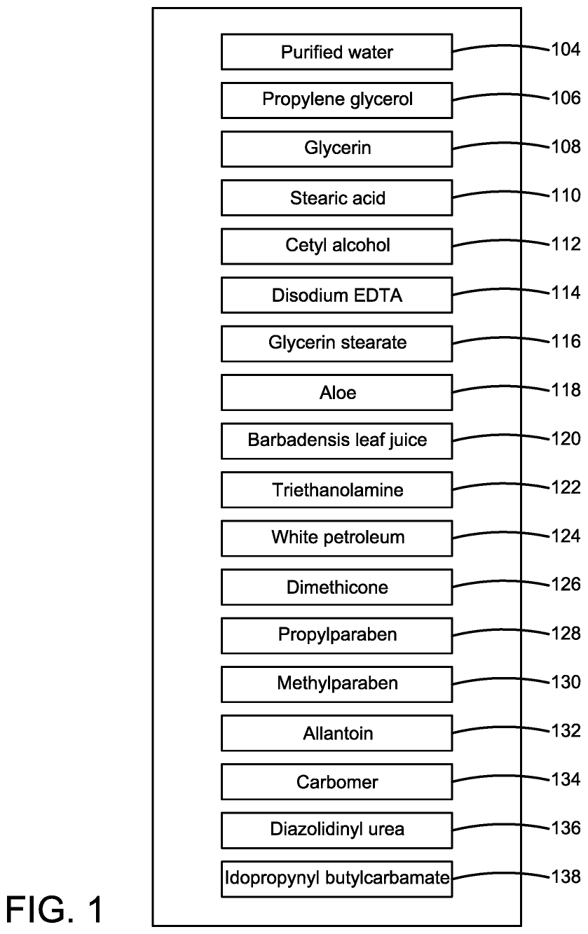

Referring initially to the drawings, FIG. 1 illustrates a graphical view of the ingredients of one potential embodiment of a lotion compound 100 of the present invention in accordance with the disclosed architecture. The compound 100 is comprised of a lotion compound for human skin. The compound 100 can be used to simultaneously moisture and sanitize the hands of a user. The compound 100 is comprised of a plurality of ingredients. Said ingredients include:
1. purified water 104
2. propylene glycerol 106
3. glycerin 108
4. stearic acid 110
5. cetyl alcohol 112
6. disodium EDTA 114
7. glyceryl stearate 116
8. Aloe 118
9. barbadensis leaf juice 120
10. triethanolamine 122
11. white petroleum 124
12. dimethicone 126
13. propylparaben 128
14. methylparaben 130
15. allantoin 132
16. carbomer 134
17. diazolidinyl urea 136
18. iodopropynyl butylcarbamate 138

In the preferred embodiment, each ingredient makes up the lotion compound 100 at 1% weight. However, the percentage weights of each ingredient may differ in various embodiments of the compound 100.

Each ingredient serves a distinct function within the compound 100. Stearic acid 110 and glyceryl stearate 116 act as emulsifying compounds. In this manner, said ingredients prevent the other ingredients from separating when forming the compound 100.

Aloe 118 and barbadensis leaf juice 120 act as soothing agents within the compound 100. Both ingredients help lighten discomfort of the skin and act in an anti-inflammatory and anti-irritant capacity. They also act to slow the aging process of skin.

Glycerin 108 (or glycerol), propylene glycerol 106, dimethicone 126, and stearic acid 110 act as a moisturizer, a humectant, and an emollient. These ingredients moisturize the skin of the user and also help the skin of the user retain water. These ingredients also make a user's skin feel smooth and soft.

Cetyl Alcohol 112 serves a variety of functions within the compound 100. First, cetyl alcohol 112 serves as an emollient to help the user's skin feel smooth. Ceytl alcohol 112 also acts as an emulsifier to blend the compound 100. In differing embodiments, the cetyl alcohol 112 used in the compound 100 can be derived from coconut, coconut oil, palm kernel oil, etc.

Triethanolamine 122 aids in setting the pH level of the compound 100. Propylparaben 128 and white petroleum 124 act as preservatives in the compound. Similarly, disodium EDTA 114, diazolidinyl urea 138, and methylparaben 130 also acts as a preservative in the compound by neutralizing metal ions within the compound 100. Iodopropynyl butylcarbamate 140 also acts as a preservative against yeast and molds.

Carbomer 134 is used within the compound 100 for consistency. Carbomer 134 converts the liquid compound into a viscous gel formula. Purified water 104 is also included within the compound 100 to dissolve other ingredients of the compound 100. Allantoin 132 may be used in the compound 100 for hydration, exfoliation, moisturizing, and soothing purposes.

The compound 100 may be any color known in the art in different embodiments. Said colors include, but are not limited to, royal blue, sunset red, sunset yellow, deep pink, grape violet, lemon yellow, super red, etc. The compound 100 may also be scented with any scent known in the art. Said scents include, but are not limited to, mango, orange, peach, grape, cherry, peppermint, pineapple, watermelon, lavender, jasmine, rose, etc.

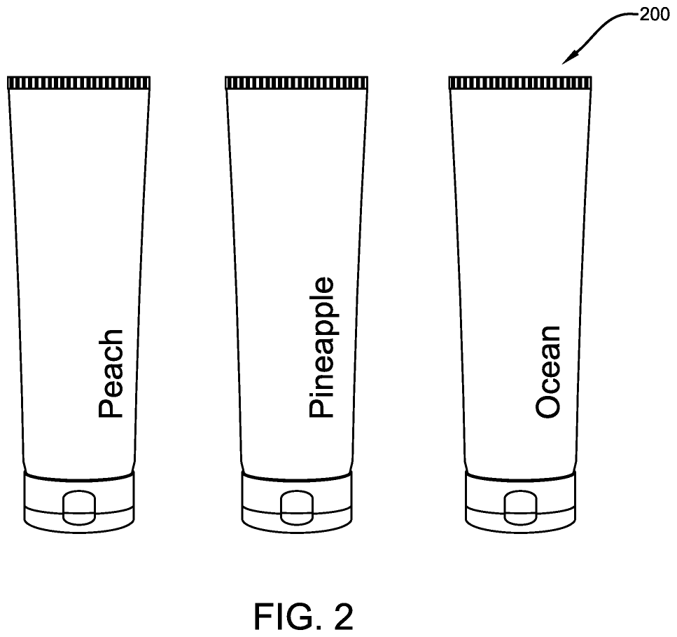

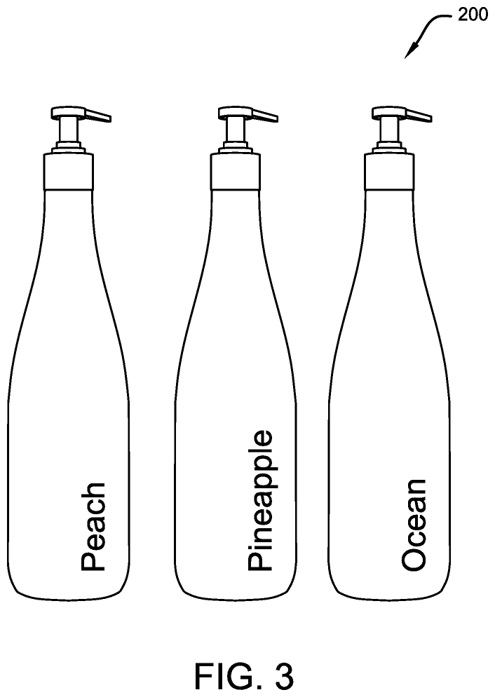

The compound 100 is also comprised of at least one container 200, as seen in FIG. 2 and FIG. 3. The container 200 houses the compound 100. The container 200 may be any type of lotion-dispensing container known in the art such as, but not limited to, a squeeze container, a push top container, a bottle, a jar, a tube, etc.

Certain terms are used throughout the following description and claims to refer to particular features or components. As one skilled in the art will appreciate, different people may refer to the same feature or component by different names. This document does not intend to distinguish between components or features that differ in name but not structure or function. As used herein “lotion compound” and “compound” are interchangeable and refer to the lotion compound 100 of the present invention.

Notwithstanding the foregoing, the lotion compound 100 of the present invention and its various components can be of any suitable size and configuration as is known in the art without affecting the overall concept of the invention, provided that they accomplish the above-stated objectives. One of ordinary skill in the art will appreciate that the size, configuration, and material of the lotion compound 100 as shown in the FIGS. are for illustrative purposes only, and that many other sizes and shapes of the lotion compound 100 are well within the scope of the present disclosure. Although the dimensions of the lotion compound 100 are important design parameters for user convenience, the lotion compound 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A lotion composition comprising:
a purified water;
a propylene glycerol;
a glycerin;
a stearic acid;
a cetyl alcohol;
a disodium EDTA;
a glyceryl stearate;
an aloe;
a barbadensis;
a leaf juice;
a triethanolamine;
a white petroleum;
a dimethicone;
a propylparaben;
a methylparaben;
an allantoin;
a carbomer;
a propylene;
a diazolidinyl urea; and
an iodopropynyl butylcarbamate.

2. The lotion composition of claim 1, wherein the purified water is at 1% weight of the lotion composition.

3. The lotion composition of claim 1, wherein the propylene glycerol is at 1% weight of the lotion composition.

4. The lotion composition of claim 1, wherein the glycerin is at 1% weight of the lotion composition.

5. The lotion composition of claim 1, wherein the stearic acid is at 1% weight of the lotion composition.

6. The lotion composition of claim 1, wherein the cetyl alcohol is at 1% weight of the lotion composition.

7. The lotion composition of claim 1, wherein the disodium EDTA is at 1% weight of the lotion composition.

8. The lotion composition of claim 1, wherein the glyceryl stearate is at 1% weight of the lotion composition.

9. The lotion composition of claim 1, wherein the aloe is at 1% weight of the lotion composition.

10. The lotion composition of claim 1, wherein the barbadensis is at 1% weight of the lotion composition.

11. The lotion composition of claim 1, wherein the leaf juice is at 1% weight of the lotion composition.

12. The lotion composition of claim 1, wherein the triethanolamine is at 1% weight of the lotion composition.

13. The lotion composition of claim 1, wherein the white petroleum is at 1% weight of the lotion composition.

14. The lotion composition of claim 1, wherein the dimethicone is at 1% weight of the lotion composition.

15. The lotion composition of claim 1, wherein the propylparaben is at 1% weight of the lotion composition.

16. The lotion composition of claim 1, wherein the methylparaben is at 1% weight of the lotion composition.

17. The lotion composition of claim 1, wherein the allantoin is at 1% weight of the lotion composition.

18. The lotion composition of claim 1, wherein each of the carbomer, the propylene, the iodopropynyl butylcarbamate and the diazolidinyl urea is at 1% weight of the lotion composition.

19. A lotion composition comprising:
a propylene glycerol;
a glycerin;
a stearic acid;
a cetyl alcohol;
a disodium EDTA;
a glyceryl stearate;
an aloe;
a barbadensis;
a leaf juice;
a triethanolamine;
a white petroleum;
a dimethicone;
a propylparaben;
a methylparaben;
an allantoin;
a carbomer;
a propylene;
a diazolidinyl urea; and
an iodopropynyl butylcarbamate.

20. A lotion composition comprising:
a purified water;
a propylene glycerol;
a glycerin;
a stearic acid;
a cetyl alcohol;
a disodium EDTA;
a glyceryl stearate;
an aloe;
a barbadensis;
a triethanolamine;
a white petroleum;
a dimethicone;
a propylparaben;
a methylparaben;
an allantoin;
a carbomer;
a propylene;
a diazolidinyl urea; and
an iodopropynyl butylcarbamate.
